(19) United States
(12) Patent Application Publication
(10) Pub. No.: US 2026/0295172 A1
(43) Pub. Date: Oct. 1, 2026

(54) SYRINGE SYSTEM AND ASSOCIATED METHODS

(71) Applicant: CLEARSTREAM TECHNOLOGIES LIMITED, Enniscorthy, County Wexford (IE)

(72) Inventors: Jordan Addison, Gilbert, AZ (US); Dylan Beyhl, Phoenix, AZ (US); Tyler Lent, Mesa, AZ (US); Ruben Ramirez, Mesa, AZ (US); Charles D. Shermer, Raleigh, NC (US); Heather A. Storm, Phoenix, AZ (US)

(73) Assignee: CLEARSTREAM TECHNOLOGIES LIMITED, Enniscorthy, County Wexford (IE)

(21) Appl. No.: 19/477,656

(22) PCT Filed: May 10, 2024
(86) PCT No.: PCT/US2024/028909

§ 371 Date: Oct. 22, 2025

Related U.S. Application Data
(60) Provisional application No. 63/501,534, filed on May 11, 2023.
(60) Provisional application No. 63/501,546, filed on May 11, 2023.

(51) Int. Cl. A61M 5/315; A61J 1/20; A61M 5/14; A61M 5/145; A61N 5/10

(52) U.S. Cl. CPC A61M 5/31596; A61J 1/2058; A61J 1/2096; A61M 5/1408; A61M 5/1452; A61N 5/10; A61N 2005/1094

(57) ABSTRACT

In one embodiment, a syringe system includes a dual barrel syringe, a dual chamber mixing cartridge, and a plurality of mixing masses. The dual barrel syringe includes a first diluent chamber, a second diluent chamber, a first port fluidically coupled to the first diluent chamber, and a second port fluidically coupled to the second diluent chamber. The dual chamber mixing cartridge is configured to mount to the dual barrel syringe and includes a first powder chamber, a second powder chamber, a first cartridge port fluidically coupled to the first powder chamber, and a second cartridge port fluidically coupled to the second powder chamber. The plurality of mixing masses are positioned within the first powder chamber and the second powder chamber.

### CROSS-REFERENCE TO RELATED APPLICATIONS

The present application claims priority to U.S. Provisional Application No. 63/501,534, entitled “MIXING SYRINGE SYSTEMS AND DEVICES, AND METHODS OF MIXING CONSTITUENT MATERIALS,” filed May 11, 2023, and U.S. Provisional Application no. 63/501,546, entitled “MIXING SYRINGE ASSEMBLIES AND METHODS OF MIXING CONSTITUENT MATERIALS,” filed May 11, 2023, the entireties of which are hereby incorporated by reference herein.

### BACKGROUND

### Field

The present disclosure generally relates to syringe systems and methods and, more particularly, syringe systems and methods for mixing constituent materials for radiation spacers.

### Technical Background

Prostate cancer is the most common non-skin cancer diagnosed in men. Radiation therapy is an excellent treatment option for prostate cancer. However, radiation exposure can cause unintended side effects in adjacent non-targeted tissue. A radiation spacer, such as a radio protective spacer, can be implanted to avoid collateral radiation and minimize injury to nearby tissues by providing a space between the target tissue and non-targeted tissues at risk.

Conventional radiation spacers may include gelling materials, which are delivered as a liquid and allowed to cure. However, constituent components of the gel may begin to gel on contact with one another, which may make delivery difficult. Moreover, constituent components may need to remain separate until time of use, otherwise they may degrade over time due to limited stability.

### SUMMARY

Embodiments of the present disclosure are directed to various syringe systems, which may maintain separation of constituent materials until time for use of the mixed constituent materials.

In one embodiment, a syringe system includes a dual barrel syringe, a dual chamber mixing cartridge, and a plurality of mixing masses. The dual barrel syringe includes a first diluent chamber, a second diluent chamber, a first port fluidically coupled to the first diluent chamber, and a second port fluidically coupled to the second diluent chamber. The dual chamber mixing cartridge is configured to mount to the dual barrel syringe and includes a first powder chamber, a second powder chamber, a first cartridge port fluidically coupled to the first powder chamber, and a second cartridge port fluidically coupled to the second powder chamber. The plurality of mixing masses are positioned within the first powder chamber and the second powder chamber.

In another embodiment, a syringe system includes a dual barrel syringe, a dual chamber mixing cartridge, and a collar. The dual barrel syringe includes a first diluent chamber, a second diluent chamber, a first port fluidically coupled to the first diluent chamber, and a second port fluidically coupled to the second diluent chamber. The dual chamber mixing cartridge includes a first powder chamber, a second powder chamber, a first cartridge port fluidically coupled to the first powder chamber, and a second cartridge port fluidically coupled to the second powder chamber. The collar is configured to receive and fluidically couple (1) the first port and the first cartridge port and (2) the second port and the first cartridge port, wherein the collar is rotatable relative to the first and second ports and first and second cartridge ports to couple the dual chamber mixing cartridge to the dual barrel syringe.

In yet another embodiment, a method of mixing constituent materials includes coupling a dual barrel syringe to a dual chamber mixing cartridge. The dual barrel syringe includes a first diluent chamber holding a first diluent, a second diluent chamber holding a second diluent, a first port fluidically coupled to the first diluent chamber, and a second port fluidically coupled to the second diluent chamber. The dual chamber mixing cartridge includes a first powder chamber holding a first powder, a second powder chamber holding a second powder, a first cartridge port fluidically coupled to the first powder chamber, and a second cartridge port fluidically coupled to the second powder chamber, and a plurality of mixing masses positioned within the first powder chamber and the second powder chamber. The method further includes advancing a plunger within at least one of the first diluent chamber or the second diluent chamber to cause the first diluent or the second diluent to enter the first powder chamber or the second powder chamber, respectively, and agitating the plurality of mixing masses within the dual chamber mixing cartridge to dissolve the first powder or the second powder within the respective first diluent or second diluent.

In yet another embodiment, a method of mixing component materials includes coupling a dual barrel syringe to a dual chamber mixing cartridge with a collar. The dual barrel syringe includes a first diluent chamber holding a first diluent, a second diluent chamber holding a second diluent, a first port fluidically coupled to the first diluent chamber, a second port fluidically coupled to the second diluent chamber. The dual chamber mixing cartridge incudes a first powder chamber holding a first powder, a second powder chamber holding a second powder, a first cartridge port fluidically coupled to the first powder chamber, and a second cartridge port fluidically coupled to the second powder chamber. The collar fluidically couples (1) the first port and the first cartridge port and (2) the second port and the first cartridge port, wherein the collar is rotatable relative to the first and second ports and first and second cartridge ports to couple the dual chamber mixing cartridge to the dual barrel syringe. The method further includes advancing a plunger within at least one of the first diluent chamber or the second diluent chamber to cause the first diluent or the second diluent to enter the first powder chamber or the second powder chamber through the collar, respectively and agitating the dual chamber mixing cartridge to dissolve the first powder or the second powder within the respective first diluent or second diluent.

Additional features and advantages of the aspects described herein will be set forth in the detailed description, which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 schematically depicts a sectional view of an illustrative syringe system including a dual chamber mixing cartridge, and an applicator, according to one or more embodiments shown and described herein;

FIG. 2 depicts a distal end view of an illustrative quarter turn connector of the dual barrel syringe of FIG. 1, according to one or more aspects shown and described herein;

FIG. 3 depicts a proximal end view of an illustrative corresponding quarter turn connector of the dual chamber mixing cartridge of FIG. 1, according to one or more aspects shown and described herein;

FIG. 4 depicts a proximal end view of an applicator, according to one or more embodiments shown and described herein;

FIG. 5A depicts connection of the dual chamber mixing cartridge of FIG. 1 in an open quarter turn configuration to the dual barrel syringe, according to one or more embodiments shown and described herein;

FIG. 5B depicts connection of the dual chamber mixing cartridge in a closed quarter turn configuration to the dual barrel syringe of FIG. 5A, according to one or more embodiments shown and described herein;

FIG. 5C depicts a sectional view of the dual chamber mixing cartridge and dual barrel syringe of FIG. 5B, according to one or more embodiments shown and described herein;

FIG. 5D depicts advancing plungers within the dual barrel syringe of FIG. 5C to cause diluents to flow into the dual chamber mixing cartridge, according to one or more embodiments shown and described herein;

FIG. 5E depicts withdrawal of the plungers of FIG. 5D to draw the resulting mixtures back in respective chambers of the dual barrel syringe, according to one or more embodiments shown and described herein;

FIG. 6A depicts a dual barrel syringe, according to one or more embodiments shown and described herein;

FIG. 6B depicts a cap of the dual barrel syringe of FIG. 6A removed, according to one or more embodiments shown and described herein;

FIG. 6C depicts a first port and a second port of the dual barrel syringe of FIG. 6A, according to one or more embodiments shown and described herein;

FIG. 7A depicts a dual chamber mixing cartridge, according to one or more embodiments shown and described herein;

FIG. 7B depicts a first cartridge port and a second cartridge portion of the dual chamber mixing cartridge of FIG. 7A, according to one or more embodiments shown and described herein;

FIG. 8A depicts a collar for coupling a dual barrel syringe to a dual chamber mixing cartridge, according to one or more embodiments shown and described herein;

FIG. 8B depicts a sectional view of the collar of the FIG. 8A, according to one or more embodiments shown and described herein;

FIG. 9A depicts the collar of FIG. 8A coupled to a dual barrel syringe, according to one or more embodiments shown and described herein;

FIG. 9B depicts a sectional view of the collar engaged with the dual barrel syringe of FIG. 9A to a dual chamber mixing cartridge, according to one or more embodiments shown and described herein;

FIG. 9C depicts a sectional view of the collar of FIG. 9B rotated to couple the dual barrel syringe to the dual chamber mixing cartridge, according to one or more embodiments shown and described herein;

FIG. 10A depicts a dual barrel syringe adjacent an applicator, according to one or more embodiments shown and described herein;

FIG. 10B depicts a sectional view of the applicator of FIG. 10A engaged with the dual barrel syringe, according to one or more embodiments shown and described here;

FIG. 10C depicts the applicator rotated relative to FIG. 10B to couple to the dual barrel syringe, according to one or more embodiments shown and described here; and

FIG. 10D schematically delivery of a radiation spacer from the applicator of FIG. 10C, according to one or more aspects shown and described herein.

### DETAILED DESCRIPTION

The present disclosure is generally directed to syringe systems for mixing a plurality of constituent materials. Moreover, the present application is directed to syringe systems configured for delivering a radiation spacer. For example, radiation spacers may be formed of one or more hydrogel materials, which may be delivered to a desired location, such as within a balloon or on its own and cured in place to block or substantially block radiation, which may be unintentionally directed toward healthy tissue instead of targeted/diseased tissue. During mixing of the hydrogel for forming a radiation spacer, multiple materials may need to be combined just prior to and/or at delivery of the radiation spacer into a target location within the body. To maintain quality of material, powder components may need to be kept dry or unconstituted prior to application. However, during use, powder components may need to be hydrated or mixed with a diluent. It may be difficult to determine proper mixing volumes, maintain sterility, and prevent unwanted early mixing using traditional methods. Embodiments of the present disclosure may provide constituent materials in prepackaged, pre-measured, ready-to use syringe assemblies, which, as will be described in greater detail herein, provide improved mixing with reduced air bubbles, maintains sterility, and/or improves delivery.

In one particular embodiment, a syringe system for mixing of constituent materials includes a dual barrel syringe including a first diluent chamber, a second diluent chamber, a first port fluidically coupled to the first diluent chamber, and a second port fluidically coupled to the second diluent chamber. The system further includes a dual chamber mixing cartridge configured to mount to the dual barrel syringe and includes a first powder chamber, a second powder chamber, a first cartridge port fluidically coupled to the first powder chamber, and a second cartridge port fluidically coupled to the second powder chamber. In some embodiments, a plurality of mixing masses may be positioned within the first powder chamber and the second powder chamber to assist in mixing constituent materials. In some embodiments, a collar may be included to receive and fluidically couple (1) the first port and the first cartridge port and (2) the second port and the first cartridge port, wherein the collar is rotatable relative to the first and second ports and first and second cartridge ports to couple the dual chamber mixing cartridge to the dual barrel syringe. Such collar may provide for quick and efficient, leak-proof coupling of components.

The systems, devices, and methods described herein are described with a quarter turn coupling mechanism that is used to couple components together for the purposes of mixing. It should be understood that the term “quarter turn” as used herein refers to a rotation that completes about one fourth of a full rotation. That is, a quarter turn rotation rotates about 90° clockwise or counterclockwise. The components described herein are referred to as “quarter turn” components because the components allow about a 90° rotation of components relative to one another. However, it should be understood that the use of quarter turn connectors is only one illustrative example, and other types of connectors (whether turning/rotating connectors, snap-in connectors, press fit connectors, etc.) are contemplated and included in the scope of the present disclosure. In particular, quarter turn connectors may be further described in International Patent Application No. PCT/US2021/023171, entitled “Multi-Component Sealant Delivery Systems Incorporating Quarter Turn Connectors,” filed Mar. 19, 2021, the entirety of which is hereby incorporated by reference.

This solution realized by the devices, systems, and methods described herein has several distinct advantages over conventional devices, systems, and methods. First, in embodiments including mixing masses, the mixing masses are contained within the dual chamber mixing cartridge instead of the dual barrel syringe, which prevents interference of the mixing masses with the hydrogel deployment, while reducing air bubbles otherwise generated by mixing processes. Second, purging excess air into the dual chamber mixing cartridge prevents inadvertent loss of hydrogel solution. If hydrogel solution is mistakenly purged into the dual chamber mixing cartridge the user can simply draw the fluid back into the dual barrel syringe. Third, in embodiments including a collar as described herein, assembly processes may be improved.

Directional terms as used herein—for example up, down, right, left, front, back, top, bottom—are made only with reference to the figures as drawn and are not intended to imply absolute orientation unless otherwise specified.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

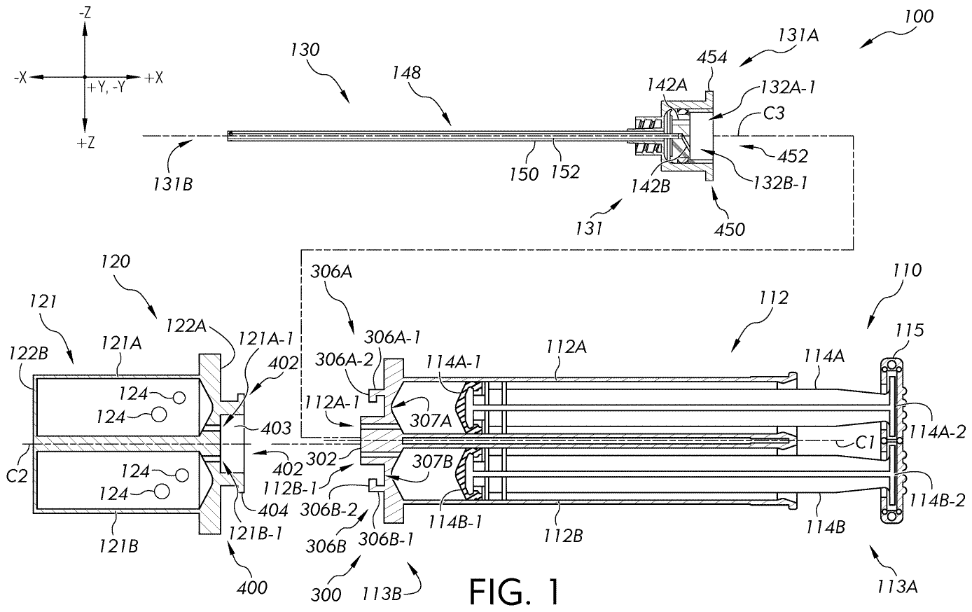

Turning now to the figures, FIG. 1 depicts an illustrative syringe system 100 for mixing constituent materials according to various embodiments. The syringe system 100, in accordance with an aspect of the present disclosure, may be for use mixing and/or delivery of a radiation spacer such as noted above.

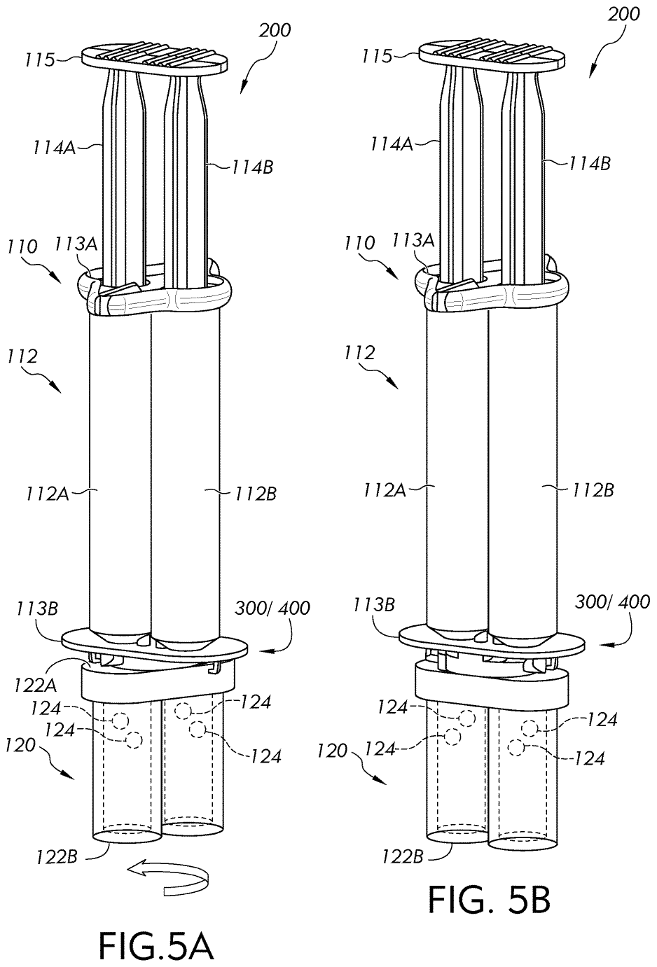

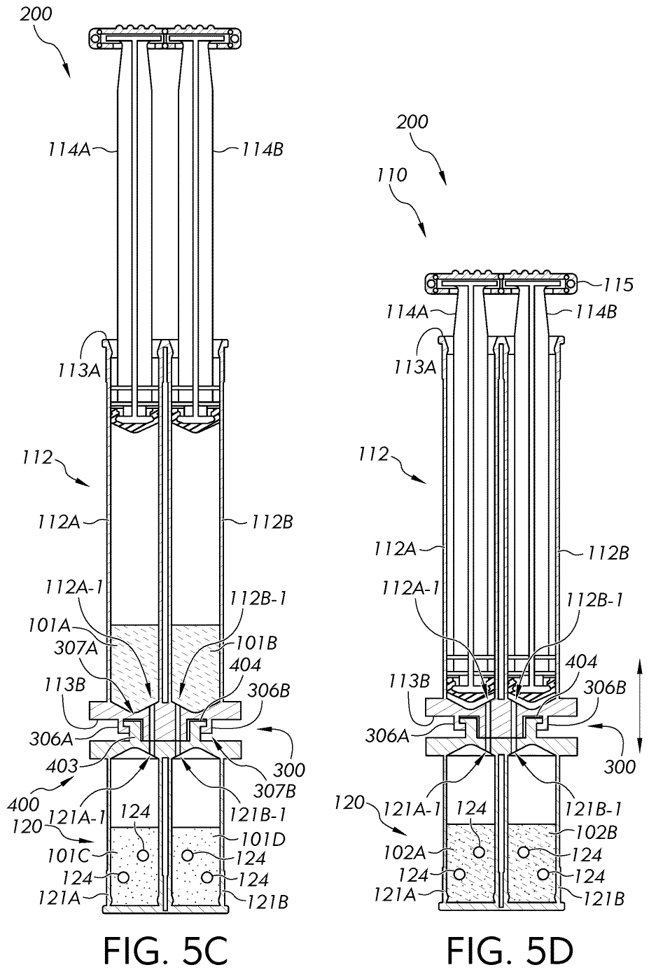

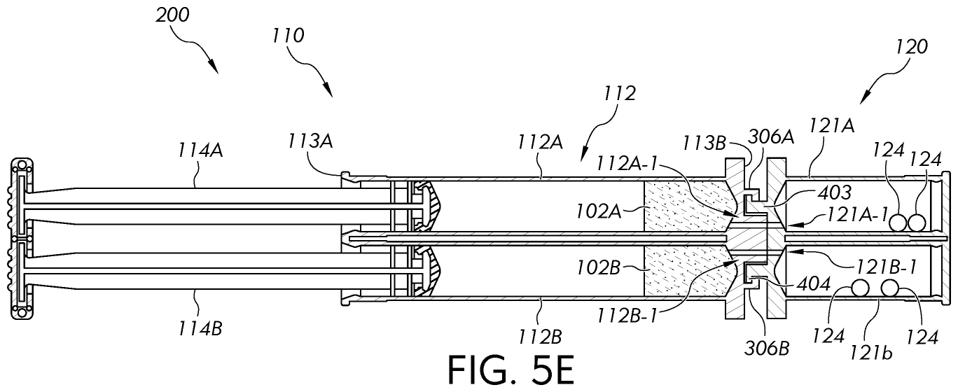

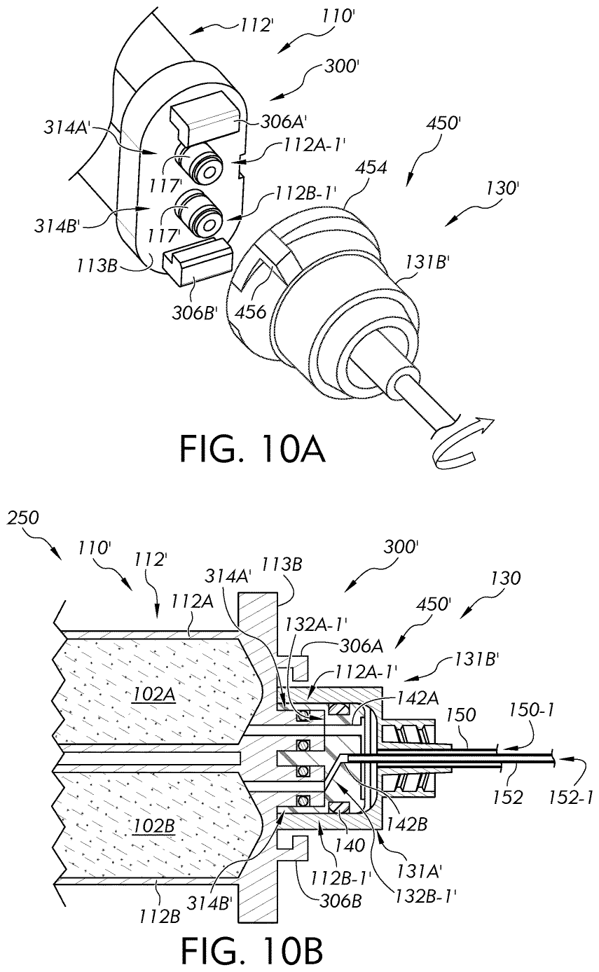

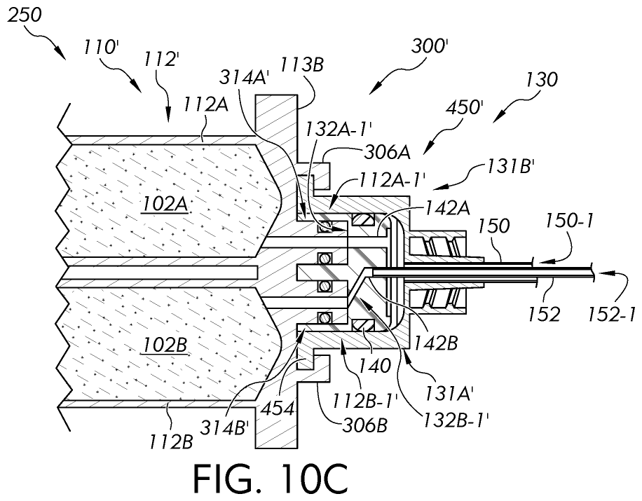

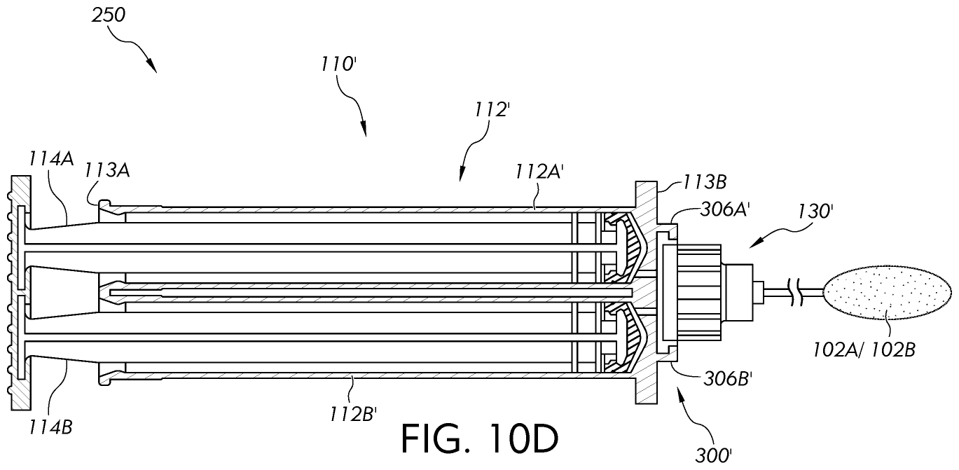

The syringe system 100 generally includes a dual barrel syringe 110 and a dual chamber mixing cartridge 120. In some embodiments, the syringe system 100 may further include an applicator 130 for delivery of a radiation spacer, for example. The various components for the syringe system 100 may be keyed via a connection system such that the dual barrel syringe 110 can be coupled to the dual chamber mixing cartridge 120 for mixing components of a multi-component material, removed from the dual chamber mixing cartridge 120, and coupled to the applicator 130 for further mixing and delivery of components, as indicated by the dashed lines in FIG. 1 and described herein. When coupled to the dual chamber mixing cartridge 120, the combination of the dual chamber mixing cartridge 120 and the dual barrel syringe 110 may be referred to herein as a mixing apparatus 200, as depicted in FIGS. 5A-5E. When coupled to the applicator 130, the combination of the applicator 130 and the dual barrel syringe 110 may be referred to herein as a delivery apparatus 250, an embodiment of which is depicted in FIG. 10A-10C.

In embodiments, the dual barrel syringe 110 may include a barrel body 112. The barrel body 112 may define a first diluent chamber 112A, a second diluent chamber 112B, a first port 112A-1 fluidically coupled to the first diluent chamber 112A, and a second port 112B-1 fluidically coupled to the second diluent chamber 112B. The dual barrel syringe 110 may further include a plunger 114A, 114B corresponding to each of the first diluent chamber 112A and the second diluent chamber 112B. In particular, the barrel body 112 may be define two separate hollow voids defining the first diluent chamber 112A and the second diluent chamber 112B. The dual barrel syringe 110 may have a proximal end 113A which provides an opening into each of the respective first diluent chamber 112A and second diluent chamber 112B for receiving the plungers 114A, 114B. At a distal end 113B opposite the proximal end 113A are positioned the first port 112A-1 and the second port 112B-1. The first port 112A-1 and the second port 112B-1 may be used for delivering material, such as to/from the dual chamber mixing cartridge 120 or to the applicator 130, as will be described in greater detail herein. As depicted, the first port 112A-1 and the second port 112B-1 may be offset from a centerline of each respective diluent chamber 112A, 112B, such as toward one another as depicted. This may be useful in providing a more compact assembly. However, in some embodiments, the first port 112A-1 and the second port 112B-1 may be aligned with the centerline of each respective diluent chamber 112A, 112B.

Referring briefly to FIG. 5C, within each respective diluent chamber 112A, 112B may be a first diluent 101A and a second diluent 101B. Diluents may be any liquid component material able to dissolve or suspend a powder component therein. For example, in some embodiments, the first diluent 101A, the second diluent 101B, or both may be saline, water, deionized water, or the like. The diluents 101A, 101B may be the same or different from one another. Moreover, the diluents 101A, 101B may be provided in the same or differing amounts.

Referring again to FIG. 1, the plungers 114A, 114B may be slidably disposed, respectively, in the first diluent chamber 112A and the second diluent chamber 112B of the barrel body 112 and may form a fluidic seal with the barrel body 112 via a seal 114A-1. 114B-1 at a distal end of each plunger 114A, 114B. For example, the seal 114A-1. 114B-1 may be a rubber or similar material conformable material for forming a fluid-tight seal with the barrel body 112. At a proximal end of each plunger 114A-1, 114B-1 may be a pusher flange 114A-2, 114B-2 for a user to engage for advancing the plunger 114A, 114B along the barrel body 112. In some embodiments, a plunger connector 115 may encapsulate both of the pusher flanges 114A-2, 114B-2 thereby synchronizing movement to the plungers 114A, 114B with one another. Such may ensure mixing and/or delivery happens concurrently.

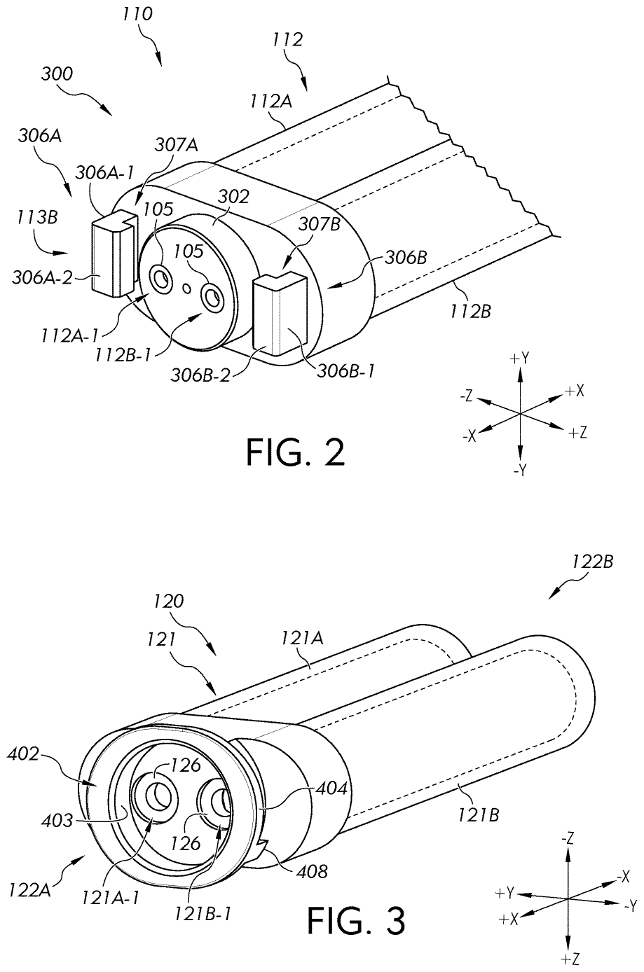

Referring to FIG. 2, the barrel body 112 of the dual barrel syringe 110 further includes a connector integrated with the distal end 113B of the dual barrel syringe 110 about the first port 112A-1 and the second port 112B-1. In the embodiments described herein, the barrel body 112 of the dual barrel syringe 110 includes a quarter turn connector 300 integrated with the distal end 113B of the dual barrel syringe 110. More specifically, as depicted in FIG. 1, the various components of the quarter turn connector 300 are integrated with the barrel body 112 such that the quarter turn connector 300 and the barrel body 112 are a single monolithic piece. However, it should be understood that this is merely illustrative and the various components of the quarter turn connector 300 may be separate pieces that are permanently or semi-permanently joined with the barrel body 112 of the dual barrel syringe 110. Further, as described herein, the quarter turn connector 300 is merely illustrative, and other connectors may be utilized in lieu of the quarter turn connector 300 without departing from the scope of the present disclosure.

The quarter turn connector 300 is generally located at the distal end 113B of the barrel body 112 such that various components of the quarter turn connector 300 are positioned adjacent to the first output port 112A-1 and the second output port 112B-1. As will be described herein, the various components of the system 100 may couple to one another via a quarter turn coupling structure. For example, the quarter turn connector 300 is generally shaped and sized to releasably interlock with a corresponding quarter turn connector 400 of the dual chamber mixing cartridge 120 and/or with a corresponding quarter turn connector 450 of the applicator 130. As will be described in greater detail herein, when the applicator 130 or the dual chamber mixing cartridge 120 is coupled to the dual barrel syringe 110 via the quarter turn connectors 300, 400, 450 thereof, the various ports thereof are aligned and sealed with the first port 112A-1 and the second port 112B-1 of the dual barrel syringe 110.

Still referring to FIGS. 1 and 2, the quarter turn connector 300 of the dual barrel syringe 110 may include a circular protrusion 302 extending distally (e.g., in the −x direction of the coordinate axes) from the distal end 113B of the dual barrel syringe 110 and a pair of coupling members (e.g., a first coupling member 306A and a second coupling member 306B) disposed radially outward of the circular protrusion 302, thereby defining a path therebetween. In the depicted embodiment, the first port 112A-1 and the second port 112B-1 are disposed within the circular protrusion 302. That is, the openings into the first chamber 112A and the second chamber 112B are located within the circular protrusion 302. In some embodiments, such as depicted in FIG. 2, sealing devices 105 (such as O-rings, or the like) may be positioned around each of the first port 112A-1, 112B-1 to assist in providing fluid-tight seals when engaged with applicator 130, or the dual chamber mixing cartridge 120.

The circular protrusion 302 is generally shaped and sized to correspond to a recess formed in the applicator 130 and the dual chamber mixing cartridge 120, as described in greater detail herein. The circular protrusion 302 may generally be disposed in or around a central area of the distal end 113B of the barrel body 112. In some embodiments, the circular protrusion 302 may be concentric with the barrel body 112 such that the center axis C1 of the barrel body 112 extends through a center of the circular protrusion 302. The distance that the circular protrusion 302 extends away from the distal end 113B of the barrel body 112 is generally a distance that corresponds to a depth of the recess formed in the applicator 130 and the dual chamber mixing cartridge 120 such that the circular protrusion 302 can be completely inserted therein, but is otherwise not limited by the present disclosure.

Referring again to FIGS. 1 and 2, the first coupling member 306A and the second coupling member 306B each extend from the distal end 113B of the barrel body 112 of the dual barrel syringe 110 and are generally shaped and sized to retain the dual chamber mixing cartridge 120 or the applicator 130 when coupled to the dual barrel syringe 110. Each of the first coupling member 306A and the second coupling member 306B may be a bayonet style coupling member, an L-beam coupling member, or the like. For example, as particularly depicted in FIG. 1, the first coupling member 306A extends distally at a particular distance from the distal end 113B of the barrel body 112, turns about 90 degrees, and extends inward toward a the center axis C1 of the barrel body 112, resulting in a first extension piece 306A-1 that extends in a direction generally parallel with the center axis C1 of the barrel body 112 (e.g., along the x-axis of the coordinate axes of FIGS. 1 and 2) and a second extension piece 306A-2 that extends in a direction that is generally perpendicular to the center axis C1 of the barrel body 112 (e.g., along the z-axis of the coordinate axes of FIGS. 1 and 2), thereby defining a pocket 307A between the distal end 113B of the barrel body 112 and the second extension piece 306A-2. Similarly, the second coupling member 306B extends distally at a particular distance from the distal end 113B of the barrel body 112, turns about 90 degrees, and extends inward toward the center axis C1 of the barrel body 112, resulting in a first extension piece 306B-1 that extends in a direction generally parallel with the center axis C1 of the barrel body 112 (e.g., along the x-axis of the coordinate axes of FIGS. 1 and 2) and a second extension piece 306B-2 that extends in a direction that is generally perpendicular to the center axis C1 of the barrel body 112 (e.g., along the z-axis of the coordinate axes of FIGS. 1 and 2), thereby defining a pocket 307B between the distal end 113B of the barrel body 112 and the second extension piece 306B-2.

As depicted in FIGS. 1 and 2, the first coupling member 306A and the second coupling member 306B are located opposite one another, radially outward of the circular protrusion 302. However, this is merely illustrative, and other locations and spacing are contemplated and included within the scope of the present disclosure. Further, while the present embodiment includes a pair of coupling members (e.g., the first coupling member 306A and the second coupling member 306B), this is also merely illustrative. That is, other amounts of coupling members are also contemplated and included within the scope of the present disclosure.

Turning now to FIGS. 1 and 3, the dual chamber mixing cartridge 120 generally includes a cartridge body 121 having a proximal end 122A and a distal end 122B spaced a distance apart from the proximal end 122A. The cartridge body 121 also defines a pair of powder chambers including a first powder chamber 121A and a second powder chamber 121B. The dual chamber mixing cartridge 120 further defines a first cartridge port 121A-1 fluidically coupled to the first powder chamber 121A and a second cartridge port 121B-1 fluidically coupled to the second powder chamber 121B. In some aspects, the first powder chamber 121A and the second powder chamber 121B are arranged in a substantially longitudinally parallel arrangement. Initially, the first powder chamber and 121A the second powder chamber 121B each hold a first powder component 101C and a second powder component 101D, such as depicted in FIG. 5C. In embodiments, the first powder chamber 121A and the second powder chamber 121B may hold different powder constituent materials, and/or differing amounts. The powder materials may be powder precursors of a desired hydrogel, such as for use as a radiation spacer. Powder materials may include, but are not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts, designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9 months, 10 months, 11 months, 12 months, 13 months, 14 months, 15 months, 16 months, 17 months, and 18 months. It should be understood that the time is merely a rough guide generally used to target appropriate formulation of the hydrogel.

In some embodiments, also located within the first powder chamber 121A and/or the second powder chamber 121B are one or more mixing masses 124 (two mixing masses in each of the first powder chamber 121A and second powder chamber 121B are depicted for illustrative purposes only). The one or more mixing masses 124 may be any suitable material to assist in mixing materials such as stainless steel, acetal resin (such as Delrin® acetal resin), plastic polycarbonate, composite, aluminum, low carbon steel, chemical-resistant PTFE, composite, titanium, moisture resistant polyethylene, magnetic material, etc. In one or more mixing masses 124, may be any shape such as spherical, cylindrical, cubic, ellipsoid, or any other regular or irregular shape. The one or more mixing masses 124 may include any number of masses such as two or more, three or more, four or more etc. The one or more mixing masses 124 may be identical to one another or different from one another. The one or more mixing masses 124 may have various sizes such as up to 7 mm in diameter, such as up to 6 mm in diameter, such as up to 5 mm in diameter, such as up to 4 mm in diameter, such as up to 3 mm in diameter, such as 7 mm or less in diameter. In various embodiments, the one or more mixing masses 124 may have designs, patterns, projections, and/or the like cut or etched into its surface, which may enhance mixing.

The first cartridge port 121A-1 of the first powder chamber 121A and the second cartridge port 121B-1 of the second powder chamber 121B may be arranged within a proximal end 122A of the cartridge body 121 of the dual chamber mixing cartridge 120. The first cartridge port 121A-1 and the second cartridge port 121B-1 are generally outputs/inputs that are aligned with other ports of other components as described herein such that the respective components initially within the powder chambers 121A, 121B can be dispensed from and/or received by (e.g., such as liquid initially within the diluent chambers 112A, 112B) the respective powder chambers 121A, 121B. In some aspects, the first cartridge port 121A-1 may be concentrically aligned with the first powder chamber 121A and the second cartridge port 121B-1 may be concentrically aligned with the second powder chamber 121B. However, in other aspects, such as the aspect depicted in FIG. 1, the first cartridge port 121A-1 may be located radially inward of a central area of the first powder chamber 121A and the second cartridge port 121B-1 may be located radially inward of a central area of the second powder chamber 121B such that the first cartridge port 121A-1 and the second cartridge port 121B-1 are closer to a center axis C2 of the cartridge body 121 of the dual chamber mixing cartridge 120 to facilitate alignment with the other components of the syringe system 100 described herein.

Still referring to FIGS. 1 and 3, the corresponding quarter turn connector 400 of the dual chamber mixing cartridge 120 includes a circular recess 402 sized to receive the circular protrusion 302 on the distal end 113B of the dual barrel syringe 110. In some embodiments the quarter turn connector 400 of the dual chamber mixing cartridge 120 may include a lip 404 extending radially outward from the circular recess 402. In some embodiments, the lip 404 may have a constant thickness in a longitudinal direction (e.g., the y direction of the depicted coordinated axes). However, in some embodiments, a portion of the lip 404 may be ramped or have a gradually increasing thickness. In some embodiments, extending, such as distally extending from the lip 404 may be a stopper projection 408, configured to engage with first coupling member 306A or the second coupling member 306B. In some embodiments, the thickness of the lip 404 may increase toward the stopper projection 408 and decrease in a direction away from the stopper projection 408. Such increase toward the stopper projection 408 may increase sealing pressure when the dual chamber mixing cartridge 120 is mounted to the dual barrel syringe 110.

The circular recess 402 is generally a recess that is defined by a wall 403 extending proximally (e.g., in the +x direction of the coordinate axes of FIG. 1) from the proximal end 122A of the cartridge body 121 of the dual chamber mixing cartridge 120. The wall 403 extends around the center axis C2 of the cartridge body 121 of the dual chamber mixing cartridge 120 to form the circular recess 402. The wall 403 may be shaped and sized such that the circular recess 402 formed thereby corresponds to the shape and size of the circular protrusion 302 of the dual barrel syringe 110. In some aspects, the wall 403 may be an extension of side walls of the cartridge body 121 of the dual chamber mixing cartridge 120.

The first and second cartridge ports 121A-1, 121B-1 may extend through the proximal end 122A of the cartridge body 121 of the dual chamber mixing cartridge 120 within the circular recess 402. That is, the circular recess 402 includes the plurality of cartridge ports 121A-1, 121B-1 therein. In some aspects, the circular recess 402 may include one or more features (e.g., additional recesses, retention pieces, channels, etc.) that are adapted to hold at least one seal (e.g., a sealing element or the like) around the plurality of cartridge ports 121A-1, 122A-2. For example, a seal 126 may be held within the circular recess 402 around the first cartridge port 121A-1 and the second cartridge port 121B-1. The seals 126 may each be any seal that allows the dual chamber mixing cartridge 120 to form a seal with the dual barrel syringe 110 when brought together as described herein such that the first cartridge port 121A-1 is joined and sealed with the first output port 112A-1 (e.g., to form a fluid coupling between the first cartridge port 121A-1 and the first output port 112A-1) and the second cartridge port 121B-1 is joined and sealed with the second output port 112B-1 (e.g., to form a fluid coupling between the second cartridge port 121B-1 and the second output port 112B-1). For example, the seals 126 may be O-rings, stadium shaped seals, oval seals, and/or the like. While a single seal is depicted herein for each port, the present disclosure is not limited to such. For example, a single seal, such as a figure eight shaped gasket or the like, may be used to individually seal the ports as described herein. However, it should be understood that the ports (e.g., the first cartridge port 121A-1 and the second cartridge port 121B-1) remain sealed from one another to avoid cross contamination of the components within the respective powder chambers 121A, 121B. It should also be understood that the term “seal” is not meant to be limiting, and may encompass any type of sealing element, sealing device, or the like.

The lip 404 is generally formed by the wall 403 that defines the circular recess 402. That is, the lip 404 generally extends radially outward from the wall 403. The lip 404 is shaped and sized to be received and retained by the pair of coupling members 306A, 306B when the corresponding quarter turn connector 400 is rotated relative to the quarter turn connector 300, as described herein. That is, the lip 404 has a first thickness (e.g., as defined along the x-axis of the coordinate axes of FIG. 1) at one or more first portions thereof, which gradually increases to a second thickness (e.g., as defined along the x-axis of the coordinate axes of FIG. 1) at one or more second portions thereof to allow for compressed holding of the lip 404 by the pair of coupling members 306A, 306B within the pockets 307A, 307B thereof. In some aspects, the lip 404 may extend around an entire periphery of the wall 403. In other aspects, the lip 404 may extend only around a portion of the periphery of the wall 403. In such aspects, a plurality of lips may be used.

As noted above, in some aspects, the corresponding quarter turn connector 400 further includes a pair of stopper projections 408 (only one depicted in FIG. 3). Each one of the pair of stopper projections 408 extends distally (e.g., in the-x direction of the coordinate axes of FIG. 3, toward the distal end 122B of the cartridge body 121) from the lip 404. Each one of the stopper projections 408 is aligned on the corresponding quarter turn connector 400 such that the pair of stopper projections 408 contact the coupling members 306A, 306B during a quarter turn rotating motion to hinder further rotational movement of the corresponding quarter turn connector 400 relative to the quarter turn connector 300 beyond a quarter rotation. It should be understood that any number of stopper projections may be used, though the number of stopper projections may generally correspond to the same number of coupling members (or less than the number of coupling members). For example, if the quarter turn connector 300 includes two coupling members 306A, 306B, the corresponding quarter turn connector 400 may include one stopper projection 408 or two stopper projections 408. In some aspects, the pair of stopper projections 408 may extend radially outward from the wall 403 and may not be connected to the lip 404.

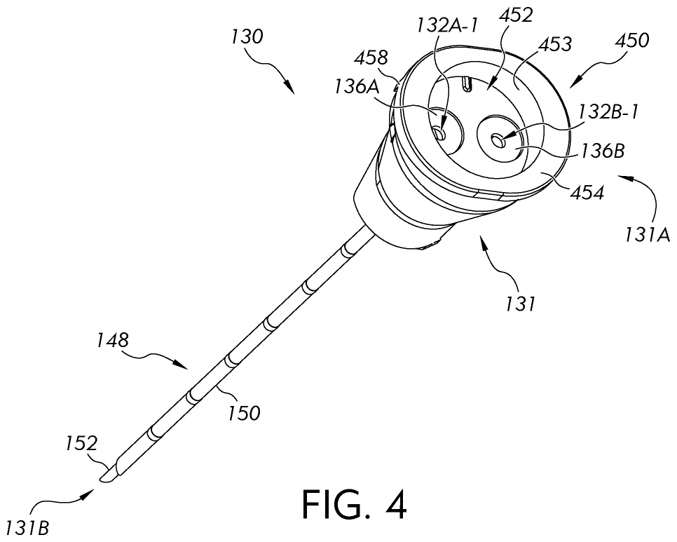

Turning now to FIGS. 1 and 4, in embodiments the applicator 130 has a proximal end 131A that extends proximally (in the +x direction of the coordinate axes of FIG. 1) and a distal end 131B that extends distally (e.g., in the −x direction of the coordinate axes of FIG. 1). The applicator 130 generally includes a hub 131 and an elongate hollow stylet 148 that extends distally (e.g., in the −x direction of the coordinate axes of FIG. 1) from hub 131 The hub 131 may be fixedly attached, e.g., through overmolding, adhesive and/or pressed fit, to the proximal end 131A of the elongate hollow stylet 148. The hub 131 includes a plurality of input ports (e.g., a first input port 132A-1 and a second input port 132B-1) of the applicator 130. The hub 131 is configured for removable connection to the dual barrel syringe 110 via the corresponding quarter turn connector 450 such that, when connected, the first input port 132A-1 of the applicator 130 is aligned and sealed with the first port 112A-1 of the dual barrel syringe 110 and the second input port 132B-1 of the applicator 130 is aligned and sealed with the second port 112B-1 of the dual barrel syringe 110, as described in greater detail herein.

The elongate hollow stylet 148 of applicator 130 is configured to facilitate fluid communication with the plurality of ports 112A-1, 112B-1 of the dual barrel syringe 110 so as to receive two combinations 102A, 102B (resulting of mixing component materials) from the dual barrel syringe 110 and direct the two combinations 102A, 102B to the distal end 131B thereof for mixing and delivery, such as generally depicted in FIGS. 10A-10C.

Still referring to FIGS. 1 and 4, in embodiments, the elongate hollow stylet 148 may be constructed, for example, of an outer elongate cannula 150 and an inner elongate cannula 152. Between the outer elongate cannula 150 and the inner elongate cannula 152 may be a first material delivery lumen 150-1 and within the inner elongate cannula 152 may be a second material delivery lumen 152-1, depicted in FIG. 10B. Accordingly, the outer elongate cannula 150 and the inner elongate cannula 152 may define two separate flow paths such that materials flowing therethrough remain separate until mixed toward a distal end 131B of the applicator 130.

Referring specifically FIG. 1, the hub 131 may further include a plurality of passageways therein for fluidly coupling the first input port 132A-1 to the first material delivery lumen 150-1 and the second input port 132B-1 to the second material delivery lumen 152-1. For example, in some aspects, a first channel 142A may couple the first input port 132A-1 to the first material delivery lumen 150-1 by extending through the hub 131 from the first input port 132A-1 to the first material delivery lumen 150-1. In another example, a second channel 142B may couple the second input port 132B-1 to the second material delivery lumen 152-1 by extending through the hub 131 from the second input port 132B-1 to the second material delivery lumen 152-1. The positioning of the first channel 142A and the second channel 142B may be dependent on a location of the first input port 132A-1 and the second input port 132B-1. For example, the first input port 132A-1 and the second input port 132B-1 are spaced to be aligned with the ports 112A-1, 112B-1 of the dual barrel syringe 110. As such, the first input port 132A-1 and the second input port 132B-1 are generally spaced the same distance radially outward from the center axis C3 of the applicator 130.

Referring to FIGS. 1 and 4, the corresponding quarter turn connector 450 of the applicator 130 includes a circular recess 452 sized to receive the circular protrusion 302 on the distal end 113B of the dual barrel syringe 110 and an applicator lip 454 extending radially outward of the circular recess 452.

The circular recess 452 is generally a recess that is defined by a wall 453 extending proximally (e.g., in the +x direction of the coordinate axes of FIG. 1) from a proximal end 131A of the applicator 130 (e.g., extending proximally from the hub 131 of the applicator 130). The wall 453 extends around the center axis C3 of the applicator 130 to form the circular recess 452. The wall 453 may be shaped and sized such that the circular recess 452 formed thereby corresponds to the shape and size of the circular protrusion 302 of the dual barrel syringe 110. In some aspects, the wall 453 may be an extension of side walls of the hub 131 of the applicator 130.

The plurality of input ports 132A-1, 132B-1 extend through the proximal end 131A of the applicator 130 within the circular recess 452. That is, the circular recess 452 includes the plurality of input ports 132A-1, 132B-1 therein. In some aspects, the circular recess 452 may include one or more features (e.g., additional recesses, retention pieces, channels, etc.) that are adapted to hold at least one seal around the plurality of input ports 132A-1, 132B-1. For example, seals 136 may be held within the circular recesses around the first input port 132A-1 the second input port 132B-1. The seals 136 may each be any seal that allows the applicator 130 to form a seal with the dual barrel syringe 110 when brought together as described herein such that the first input port 132A-1 is joined and sealed with the first port 112A-1 (e.g., to form a fluid coupling between the first input port 132A-1 and the first port 112A-1) and the second input port 132B-1 is joined and sealed with the second port 112B-1 (e.g., to form a fluid coupling between the second input port 132B-1 and the second port 112B-1). For example, the seals may be O-rings, stadium shaped seals, oval seals, and/or the like. While a single seal is depicted herein for each port, the present disclosure is not limited to such. For example, a single seal, such as a figure eight shaped gasket or the like, may be used to individually seal the ports as described herein. However, it should be understood that the ports (e.g., the first input port 132A-1 and the second input port 132B-1) remain sealed from one another to avoid premature combining of components prior to reaching the distal chamber.

Still referring to FIGS. 1 and 4, the applicator lip 454 is generally formed by the wall 453 that defines the circular recess 452. That is, the applicator lip 454 generally extends radially outward from the wall 453. The applicator lip 454 is shaped and sized to be received and retained by the pair of coupling members 306A, 306B when the corresponding quarter turn connector 450 is rotated relative to the quarter turn connector 300, as described herein. In embodiments, the applicator lip 454 has a first thickness (e.g., as defined along the x-axis of the coordinate axes of FIG. 1) at one or more first portions thereof, which gradually increases to a second thickness (e.g., as defined along the x-axis of the coordinate axes of FIG. 1) at one or more second portions thereof to allow for compressed holding of the applicator lips 454 by the pair of coupling members 306A, 306B within the pockets 307A, 307B thereof. In some aspects, the applicator lip 454 may extend around an entire periphery of the wall 453. In other aspects, the applicator lip 454 may extend only around a portion of the periphery of the wall 453. In such aspects, a plurality of applicator lips may be used.

In some aspects, the corresponding quarter turn connector 450 further includes a pair of stopper projections 458 (only one depicted in FIG. 4). Each one of the pair of stopper projections 458 extends distally (e.g., in the −x direction of the coordinate axes of FIG. 4B, toward the distal end 131B of the applicator 130) from the applicator lip 454. Each one of the pair of stopper projections 458 is aligned on the corresponding quarter turn connector 450 such that the pair of stopper projections 456 contact the coupling members 306A, 306B during a quarter turn rotating motion to hinder further rotational movement of the corresponding quarter turn connector 450 relative to the quarter turn connector 300 beyond a quarter rotation. It should be understood that any number of stopper projections may be used, though the number of stopper projections generally corresponds to the same number of coupling members (or less than the number of coupling members). For example, if the quarter turn connector 300 includes two coupling members 306A, 306B, the corresponding quarter turn connector 450 may include one stopper projection 456 or two stopper projections 456. In some aspects, the pair of stopper projections 456 may extend radially outward from the wall 453 and may not be connected to the applicator lip 454.

In an embodiment, the features of the quarter turn connectors 300, 400, 450 may advantageously result in a structure that allows a user to quickly connect and subsequently disconnect components (e.g., the dual barrel syringe 110 with the dual chamber mixing cartridge 120 and the applicator 130) in such a manner that a user can easily confirm that components are correctly sealed and aligned with one another to ensure the correct materials held within are mixed together and then subsequently delivered to a site on a subject.

As previously noted, it should be understood that the various quarter turn connectors described herein are merely illustrative and other connectors are also contemplated and included within the scope of the present disclosure.

Turning now to FIGS. 5A-5F, an illustrative method of mixing constituent materials is depicted using the dual barrel syringe 110 and dual chamber mixing cartridge 120 described above. For example, as shown in FIGS. 5A-5B, the dual barrel syringe 110 and the dual chamber mixing cartridge 120 are coupled together, such as, for example, via the quarter turn connectors 300, 400, to form the mixing apparatus 200. In particular, FIG. 5A depicts the dual barrel syringe 110 engaged with the dual chamber mixing cartridge 120 but quarter-turned out of alignment such that the dual chamber mixing cartridge 120 is not held in place by the dual barrel syringe 110. That is, the coupling members 306A, 306B are not engaged with the lip 404. FIG. 5B depicts the dual chamber mixing cartridge 120 quarter-turned into alignment. In such configuration, and as best depicted in the sectional view of FIG. 5C, the lip 404 is engaged with the coupling members 306A, 306B, in respective pockets 307A, 307B such that the dual chamber mixing cartridge 120 may not be pulled longitudinally away from the dual chamber mixing cartridge 120. Once joined in this manner, the various ports are aligned and sealed to one another to allow communication therebetween. In particular, the first port 112A-1 is aligned with the first cartridge port 121A-1 and the second port 112B-1 is aligned with the second cartridge port 121B-1. In some embodiments, and as described in greater detail below, a dual barrel syringe may be coupled to the dual chamber mixing cartridge via a collar. Accordingly, in such embodiments, methods may include coupling the dual barrel syringe 110 to the dual chamber mixing cartridge 120 via a collar as described in greater detail below.

In the depicted embodiment, a first diluent 101A is positioned within the first diluent chamber 112A, a second diluent 101B is positioned within the second diluent chamber 112B, a first powder component 101C is positioned within the first powder chamber 121A, and a second powder component 101D is positioned within the second powder chamber 121B. For example, the first powder component 101C may include albumin and the second powder component 101D may include PEG. Other combinations are contemplated and possible.

When desired and as depicted in FIG. 5D, a user may compress the plungers 114A, 114B to cause the first diluent 101A to enter the first powder chamber 121A from the first diluent chamber 112A and the second diluent 101B to enter the second powder chamber 121B from the second diluent chamber 112B. In this way, the first and second diluents 101A, 101B remain isolated from one another. In embodiments, the mixing apparatus 200 may then be agitated (such as up/down, side to side, and/or swirled) to cause the mixing masses 124 (if present) to swirl or otherwise move within the first powder chamber 121A and the second powder chamber 121B to cause the powder components 101C, 101D, and respective liquid components 101A, 101B to become thoroughly mixed resulting in a first combination 102A within the first powder chamber 121A and a second combination 102B within the second powder chamber 121B. In some embodiments, the plungers 114A, 114B may be repeatedly pulled and depressed to further mix the respective combinations 102A, 102B. Once mixing is completed, as depicted in FIG. 5E, the plungers 114A, 114B may be withdrawn, to withdraw respective combinations 102A, 102B back in to the respective first and second diluent chambers 112A, 112B while keeping the respective combinations 102A, 102B isolated from one another. In some embodiments, gas may be purged back into the dual chamber mixing cartridge 120. When purging gas, if some of the first combination 102A and/or the second combination 102B were to flow back into the first powder chamber 121A or the second powder chamber 121B, such may be withdrawn again using the plungers 114A, 114B. After the dual chamber mixing cartridge 120 is removed, the applicator 130 may be assembled to the dual barrel syringe in the substantially same manner as the dual chamber mixing cartridge 120.

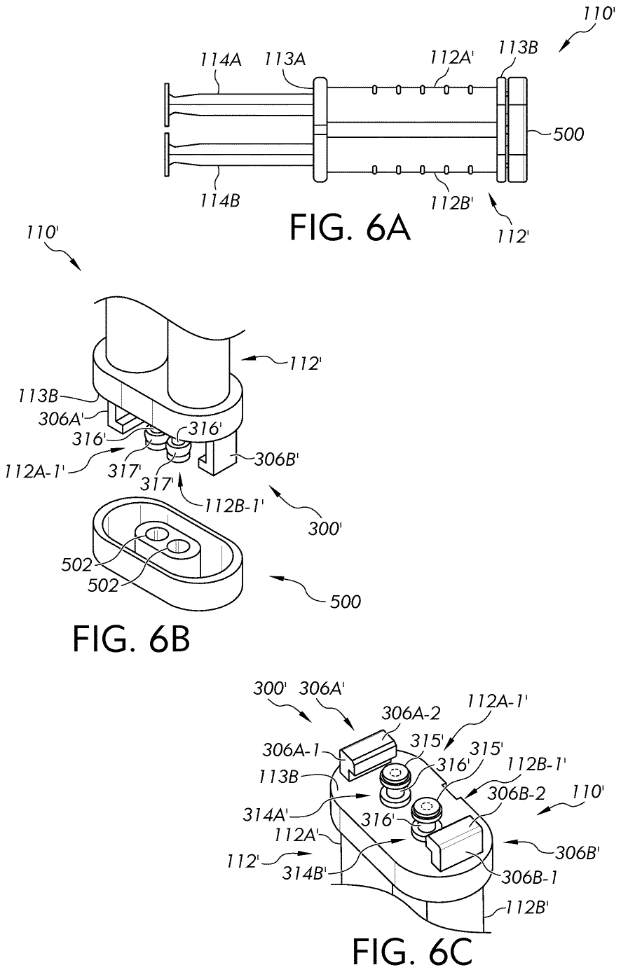

Referring now to FIG. 6A-9B an alternative embodiment of the dual barrel syringe 110′ and the dual chamber mixing cartridge 120′ are generally depicted. The embodiment is substantially similar to that described above accordingly, description of like components above applies in the present embodiment unless otherwise indicated or apparent. Accordingly, such description will not be repeated in detail.

Referring specifically to FIGS. 6A-6C, as above, the dual barrel syringe 110′ may generally include a barrel body 112′ that defines a first diluent chamber 112A′, a second diluent chamber 112B′, a first port 112A-1′ fluidically coupled to the first diluent chamber 112A′, and a second port 112B-1′ fluidically coupled to the second diluent chamber 112B′. Further the dual barrel syringe 110′ may include a quarter turn connector 300′ substantially similar to the quarter turn connector 300 described above. In particular, the quarter turn connector 300′ includes a first coupling member 306A′ and a second coupling member 306B′ substantially identical in features to coupling members 306A, 306B described above. However, in the present embodiment, the quarter turn connector 300′ does not include a circular protrusion between the first coupling member 306A′ and the second coupling member 306B′. Instead, the first port 112A-1′ and the second port 112B-1′ extend adjacent in the distal direction in a protruding fashion. In such embodiments, the first port 112A-1′ may be defined by a first port column 314A′ and the second port 112B-1′ may include a second port column 314B′. The first port column 314A′ and the second port column 314B′ may include a reduced diameter neck region 316′, within which an O-ring or similar sealing device 317′, such as described above, may be positioned. The sealing device 317′ may assist in providing a fluid-tight seal when engaged with a collar 600, described in greater detail below, or applicator.

In some embodiments, such as depicted in FIG. 6A, a cap 500 may engage the distal end 113B of the dual barrel syringe 110′. For example, the cap 500 may extend over and seal against the first port column 314A′ and the second port column 314B′, thereby preventing material leakage from respective ports 112A-1′, 112B-1′. In some embodiments, the cap 500 may cover the first and second coupling members 306A′, 306B′. As depicted in FIG. 6B, the cap 500 may include recesses 502 formed therein that is sized to receive port columns 314A′, 314B′ and engage the corresponding sealing devices 317′ to provide a fluid tight seal. Such may ensure integrity of the fluid held within the first diluent chamber 112A′ and the second diluent chamber 112B′.

Referring to FIG. 6C, in some embodiments, in addition to the cap 500 or in lieu of the cap 500, the respective ports 112A-1′, 112B-1′ may be sealed via a membrane 315′ (e.g., foil, plastic or the like). The membrane 315′ may be adhered or welded, for example, to the first port column 314A′ and the second port column 314B′ to seal the respective first and second ports 112A-1′, 112B-1′. Though two membranes are depicted, it is contemplated that a single membrane may cover both the first port 112A-1′ and the second port 112B-1′. Prior to use, the membrane 315′ may be peeled away or burst under pressure by advancing the plungers 114A, 114B within the first and second diluent chambers 112A′, 112B′. In some embodiments, instead of a membrane 315′ individual caps may extend over the first port and the second port 112A-1′, 112B-1′. It is noted that while membranes 315′ are only depicted with respect to the present embodiment, the above-described ports 112A-1′, 112B-1′ may also be covered by membranes prior mixing of component materials.

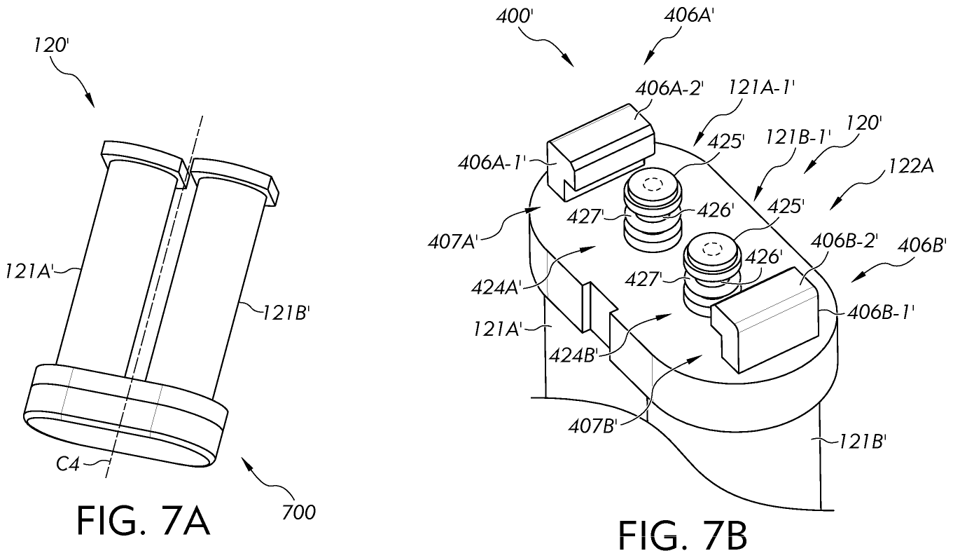

Referring now to FIGS. 7A-7B, an embodiment of a dual chamber mixing cartridge 120′ is generally depicted. The dual chamber mixing cartridge 120′ is similar to the dual chamber mixing cartridge 120 described above. Accordingly, the above description applies to the present embodiment unless otherwise described or apparent. For example, the dual chamber mixing cartridge 120′ includes a first powder chamber 121A′, a second powder chamber 121B′, a first cartridge port 121A-1′ fluidically coupled to the first powder chamber 121A′, and a second cartridge port 121B-1′ fluidically coupled to the second powder chamber 121B′. It is noted that in the depicted embodiment, a void is shown between the first powder chamber 121A′ and the second powder chamber 121B′. However, there need not be a void.

Another difference in the depicted embodiment is that the dual chamber mixing cartridge 120′ does not include a circular recess or lip. Instead, the dual chamber mixing cartridge 120′ includes a quarter turn connector 400′ substantially identical to that of the dual barrel syringe 110′. In particular, the dual chamber mixing cartridge 120′ has a first coupling member 406A′ and a second coupling member 406B′. Additionally, the first cartridge port 121A-1′ and the second cartridge port 121B-1′ extend adjacent to one another in a protruding fashion. In such embodiments, the first cartridge port 121A-1′ may be defined by a first cartridge port column 424A′ and the second cartridge port 121B-1′ may be defined by a second cartridge port column 424B′. The first cartridge port column 424A′ and the second cartridge port column 424B′ may include a reduced diameter neck region 426′, within which an O-ring or similar sealing device 427′, such as described above, may be positioned. As will be described the sealing device 427′ may assist in providing a fluid-tight seal when engaged with a collar 600, described in greater detail below.

The first and second coupling member 406A′, 406B′ may be positioned opposite one another across the first cartridge port column 424A′ and the second cartridge port column 424B′. Similar to the coupling members 306A, 306B, the first coupling member 406A′ and the second coupling member 406B′ each extend from the proximal end 122A of the dual chamber mixing cartridge 120′ and are generally shaped and sized to retain the collar 600 described in further detail below. Each of the first coupling member 406A′ and the second coupling member 406B′ may be a bayonet style coupling member, an L-beam coupling member, or the like. For example, as particularly depicted in FIG. 7B, the first coupling member 406A′ extends proximally at a particular distance from the proximal end 122A of the dual chamber mixing cartridge 120′, turns about 90 degrees, and extends inward toward a of the dual chamber mixing cartridge 120′, resulting in a first extension piece 406A-1′ that extends in a direction generally parallel with the center axis C4 of the dual mixing chamber cartridge 120′ and a second extension piece 406A-2′ that extends in a direction that is generally perpendicular to the center axis C4, thereby defining a pocket 407A′ between the proximal end 122A and the second extension piece 406A-2′. Similarly, the second coupling member 406B′ extends distally at a particular distance from the proximal end 122A, turns about 90 degrees, and extends inward toward the center axis C4, resulting in a first extension piece 406B-1′ that extends in a direction generally parallel with the center axis C4 and a second extension piece 406B-2′ that extends in a direction that is generally perpendicular to the center axis C4, thereby defining a pocket 407B′ between the proximal end 122A and the second extension piece 406B-2′.

While the present embodiment includes a pair of coupling members (e.g., the first coupling member 406A′ and the second coupling member 406B′), this is merely illustrative. That is, other amounts of coupling members are also contemplated and included within the scope of the present disclosure.

In some embodiments, such as depicted, a cap 700, which may be substantially identical to the cap 500, may engage the proximal end 122A of the dual chamber mixing cartridge 120′. For example, the cap 700 may extend over and seal against the first cartridge port 121A-1′ and the second cartridge port 121B-1′. In some embodiments, the cap 700 may cover the first and second coupling members 406A′, 406B′. As with the cap 500, the cap 700 may include recesses formed therein that are sized to receive the first cartridge port column 424A′ and the second cartridge port column 424B′ and engage the corresponding sealing devices 427′ to provide a fluid tight seal. Such may ensure integrity of the powder held within the first powder chamber 121A and the second powder chamber 121B.

Referring to FIG. 7B, in some embodiments, in addition to the cap 700 or in lieu of the cap 700, the respective cartridge ports 121A-1′, 121B-1′ may be sealed via a membrane 425′ (e.g., foil, plastic or the like). The membrane 425′ may be adhered or welded, for example, to the first cartridge port column 424A′ and the second cartridge port column 424B′ to seal the respective first and second cartridge ports 121A-1′, 121B-1′. Though two membranes are depicted, it is contemplated that a single membrane may cover both the first cartridge port 121A-1′ and the second cartridge port 121B-1′. Prior to use, the membrane 425′ may be peeled away or burst under pressure by advancing the plungers 114A, 114B when coupled to the dual barrel syringe 110′. In some embodiments, instead of a membrane 425′ individual caps may extend over the first and second cartridge ports 121A-1′, 121B-1′. It is noted that while membranes 425′ are only depicted with respect to the present embodiment, the above-described cartridge ports 121A-1, 121B-1 may also be covered by membranes 425′ prior mixing of component materials.

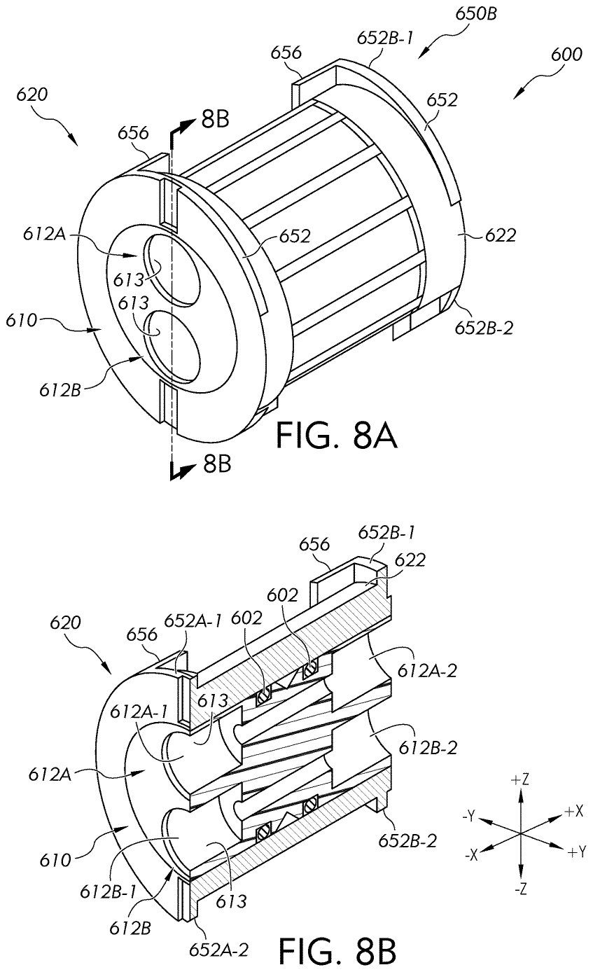

Referring now to FIGS. 8A and 8B, the collar 600 is depicted. The collar 600 receives and rotates to fluidically couple the first port 112A-1′ to the first cartridge port 121A-1′ and the second port 112B-1′ to the second cartridge port 121B-1′. In particular, the collar may include an internal body 610 and an external housing 620. The a portion of the collar 620, such as the external housing 620 or portion thereof may be rotatable relative to the internal body 610.

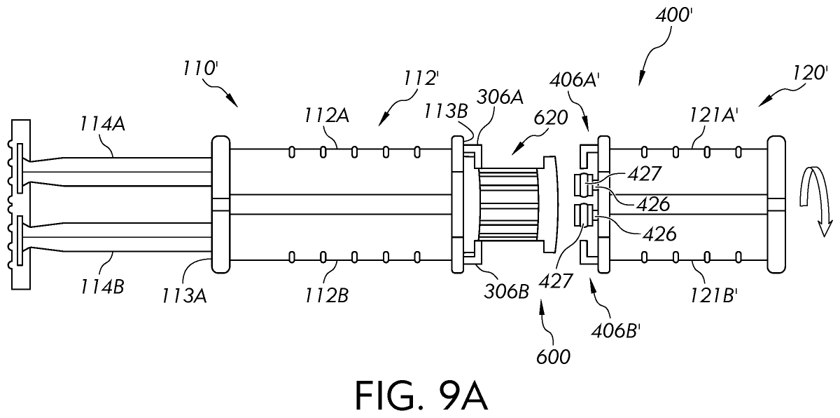

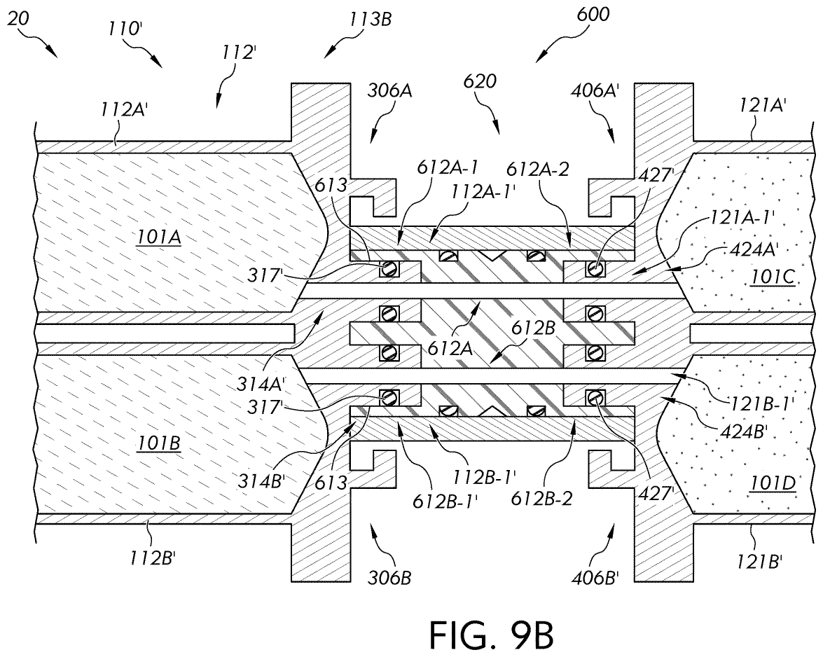

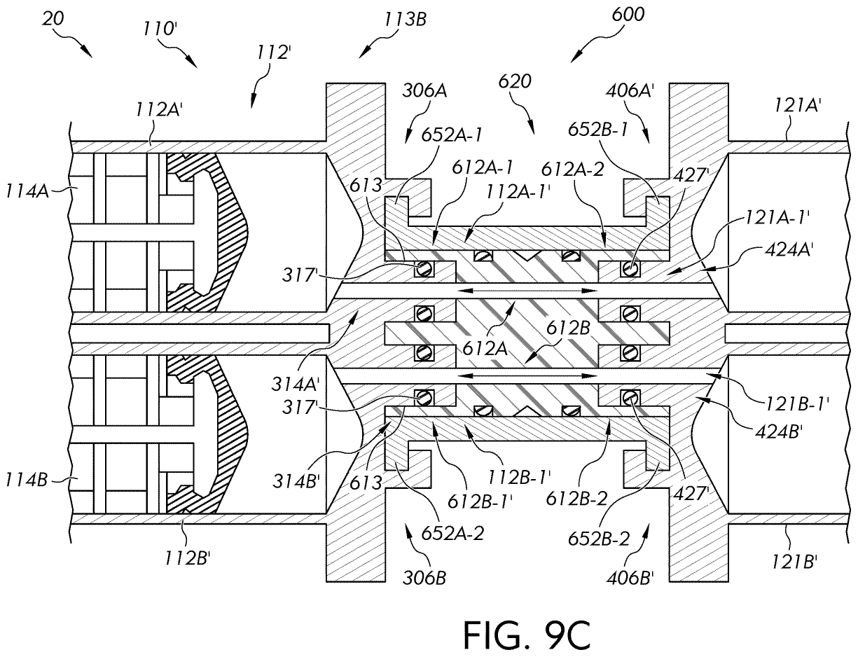

The internal body 610 may be formed of any suitable material, e.g., plastic, rubber, metal, glass, ceramic, etc., may define a first communication path 612A and a second communication path 612B. The first communication path 612A may include a first receiving port 612A-1 at a first end and a second receiving port 612A-2 at an opposite end. The first receiving port 612A-1 and the second receiving port 612A-2 may be sized to receive the first port column 314A′ and the first cartridge port column 424A′ to provide a fluid path therebetween, such as depicted in FIGS. 9B and 9C. In embodiments, the sealing devices 317′, 427′ may seal against an internal wall 613 of the internal body 610 thereby preventing component material leakage. Similarly, the second communication path 612B may include a first receiving port 612B-1 at the first end and a second receiving port 612B-2 at the opposite end. The first receiving port 612B-1 and the second receiving port 612B-2 may be sized to receive the second port column 314B′ and the second cartridge port column 424B′ to provide a fluid path therebetween, such as depicted in FIGS. 9B and 9C. In embodiments, the sealing devices 317′, 427′ may seal against an internal wall 613 of the internal body 610 thereby preventing component material leakage.

The external housing 620 may be formed of any suitable material, e.g., plastic, rubber, metal, glass, ceramic, etc. The external housing 620 may circumscribe the internal body 610 and rotate relative thereto. For example, between the external housing 620 and the internal body 610 may be bearings 602, such as ball bearings, ring bearings, etc., or a sealing device, such as an O-ring. Though two bearings are depicted, any number of bearings 602 may be included. Similar to quarter turn connectors 400 and 450, the external housing 620 includes a first quarter turn connector 650A at a first end and a second quarter turn connector 650B at a second end. The first quarter turn connector 650A and the second quarter turn connector 650B may include a plurality of tabs 652 similar to the lips described above.

The tabs 652 may be generally formed by a wall 622 of the external housing 620, which houses the internal body 610. That is, the tabs 652 may generally extends radially outward from the wall 622. Each tab 652 is shaped and sized to be received and retained by the pair of coupling members 306A′, 306B′ of quarter turn connector 300′ or the coupling members 406A′, 406B′ of quarter turn connector 400′ is rotated relative to the quarter turn connector 300′, as described herein. As with the lips described above, in embodiments, the tabs 652 may have a ramped profile. For example, each tab 652 may have a first thickness (e.g., as defined along the y-axis of the coordinate axes of FIGS. 8A, 8B) at one or more first portions thereof, which gradually increases to a second thickness at one or more second portions thereof to allow for compressed holding of the tab 652 by a pair of coupling members within respective pockets thereof. For example, the plurality of tabs may include a first cartridge engagement tab 652B-1 to be engaged by the first cartridge coupling member 406A′ and a second cartridge engagement tab 652B-2 to be engaged by the second cartridge coupling member 406B′. Similarly, the plurality of tabs may include a first barrel engagement tab 462A-1 configured to be engaged by the first coupling member 306A′ and a second barrel engagement tab 652A-2 configured to be engaged by the second coupling member 306B′. In some embodiments, extending from the tabs 652 may be a stopper projection 656, similar to stopper projections described above. Each stopper projection 656 may be configured to engage with a corresponding coupling member. In some embodiments, the thickness of the tab 652 may increase toward the stopper projection 656 and decrease in a direction away from the stopper projection 656. Such increase toward the stopper projection 656 may increase sealing pressure as described in embodiments above.

FIGS. 9A-9C illustrate the collar 600 coupling the dual barrel syringe 110′ to the dual chamber mixing cartridge 120′. In such embodiments, the first and second ports 112A-1′, 112B-1′(e.g., first and second port columns 314A′, 314B′ are arranged within respective the first receiving ports 612A-1, 612B-1 and the first and second cartridge port columns 424A′, 424B′ are arranged within respective second receiving ports 612A-2, 612B-2. The seals 317′, 427′ may engage the internal walls 613 to provide a fluid tight seal therewith. The outer housing 620 may be rotated, relative to the inner housing 610 and respective ports, such as by a quarter turn to lock respective tabs 652 beneath respective coupling members of the dual barrel syringe 110′ and the dual chamber mixing cartridge 120′.

Once attached, the plungers 114A, 114B may be advanced to move the diluent from the diluent chambers 112A′, 112B′ into the respective powder chambers 121A′, 121B′ through the collar 600. Similar to embodiments above, the mixing apparatus 200 may then be agitated to cause the mixing masses 124 (if present) and component materials to swirl or otherwise move within the first powder chamber 121A′ and the second powder chamber 121B′ to cause the powder components, and respective liquid components to become thoroughly mixed resulting in a first combination 102A within the first powder chamber 121A′ and a second combination 102B within the second powder chamber 121B′, as described above. In some embodiments, the plungers 114A, 114B may be repeatedly pulled and depressed to further mix the respective combinations 102A, 102B. Once mixing is completed, the plungers 114A, 114B may be withdrawn, to withdraw respective combinations 102A, 102B back in to the respective first and second diluent chambers 112A′, 112B′ while keeping the respective combinations 102A, 102B isolated from one another. In some embodiments, gas may be purged back into the dual chamber mixing cartridge 120 through the collar. Once sufficiently combined and moved back into the respective first diluent chamber 112A′ and the second diluent chamber 112B′, the collar 600 may be twisted (e.g., quarter turned in the opposite direction) to be removed along with the dual chamber mixing cartridge 120′.

FIGS. 10A-10D illustrate an applicator 130′ similar to the applicator 130 described above. Accordingly, the above description is applicable unless otherwise noted. In particular, in the present embodiment, the hub 131′ includes an inner portion 131A′, defining the plurality of input ports 132A-1′, 132B-1′ and an outer portion 131B′ which is rotatable relative to the inner portion 131A′. The outer portion 131B′ may be may fixedly attached to the outer elongate cannula 150 while the inner portion 131A′ is fixedly attached to the inner elongate cannula 152, such as via over molding, adhesives, press-fits, or the like. A bearing 140 (e.g., ball bearings, ring bearing, etc.) or a sealing device, such as an O-ring, may support rotation of the outer portion 131B′ relative to the inner portion 131A′. Accordingly, during rotation, the outer elongate cannula 150 may rotate relative to the inner elongate cannula 152. Though a single bearing is depicted, any number of bearings are contemplated and possible.

The quarter turn connector 450′ may be much the same as quarter turn connector 450 described above. However, in the present embodiment, the inner portion 131A′ includes two circular recesses 131A-1′, 131A-2′ for receiving the first port column 314A′ and the second port column 314B′ and sealing thereto, such as via the seals 315′. Moreover, the applicator lip 454 and stopper projections 456 are mounted to the outer portion 131B′.

Accordingly, when assembled, inner portion 131A′; receives the first port column 314A′ within the first circular recess 131A-1′ and the second port column 314B′ within the second circular recess 131A-2, depicted in FIG. 10B.′ The outer housing 131B′ may then be rotated to lock the applicator lips 454 to respective first and second coupling member 306A, 306B, as described above, depicted in FIG. 10C. Accordingly, respective mixed combinations 102A, 102B may remain separate as they flow out of the dual barrel syringe 110′ (via pressure from plungers 114A, 114B) through hub 131 and the first material delivery lumen 150-1 and the second material delivery lumen 150-2 to combine at a target delivery site, as illustrated in FIG. 10C.

Embodiments of the present disclosure may be further described with respect to the following numbered clauses:
1. A syringe system comprising: a dual barrel syringe comprising a first diluent chamber, a second diluent chamber, a first port fluidically coupled to the first diluent chamber, and a second port fluidically coupled to the second diluent chamber; a dual chamber mixing cartridge configured to mount to the dual barrel syringe and comprising a first powder chamber, a second powder chamber, a first cartridge port fluidically coupled to the first powder chamber, and a second cartridge port fluidically coupled to the second powder chamber; and a plurality of mixing masses positioned within the first powder chamber and the second powder chamber.
2. The syringe system of any preceding clause, wherein the dual barrel syringe and the dual chamber mixing cartridge couple to one another via a quarter turn coupling structure.
3. The syringe system of any preceding clause, further comprising a collar configured to receive and fluidically couple (1) the first port and the first cartridge port and (2) the second port and the first cartridge port.
4. The syringe system of any preceding clause, further comprising an applicator configured to mount to the first port and the second port of the dual barrel syringe.
5. A syringe system comprising: a dual barrel syringe comprising a first diluent chamber, a second diluent chamber, a first port fluidically coupled to the first diluent chamber, and a second port fluidically coupled to the second diluent chamber; a dual chamber mixing cartridge comprising a first powder chamber, a second powder chamber, a first cartridge port fluidically coupled to the first powder chamber, and a second cartridge port fluidically coupled to the second powder chamber; and a collar configured to receive and fluidically couple (1) the first port and the first cartridge port and (2) the second port and the first cartridge port, wherein the collar is rotatable relative to the first and second ports and first and second cartridge ports to couple the dual chamber mixing cartridge to the dual barrel syringe.
6. The syringe system of any preceding clause, wherein: the dual barrel syringe comprises a first coupling member and a second coupling member; and the collar comprises a first tab configured to be engaged to the first coupling member and a second tab configured to be engaged to the second coupling member.
7. The syringe system of any preceding clause, wherein the dual chamber mixing cartridge comprises a first cartridge coupling member and a second cartridge coupling member; and the collar comprises a first tab configured to be engaged to the first cartridge coupling member and a second tab configured to be engaged to the second cartridge coupling member.
8. The syringe system of any preceding clause, wherein the collar comprises an internal body and an external housing, wherein the external housing rotates relative to the internal body.
9. The syringe system of any preceding clause, wherein the collar comprises a plurality of ramped engagement tabs for engaging with a corresponding coupling members of the dual barrel syringe and/or the dual chamber mixing cartridge.
10. The syringe system of any preceding clause, further comprising a plurality of mixing masses positioned within the first powder chamber and the second powder chamber.
11. The syringe system of any preceding clause, further comprising an applicator configured to mount to the first port and the second port of the dual barrel syringe.
12. A method of mixing constituent materials, the method comprising: coupling a dual barrel syringe to a dual chamber mixing cartridge, wherein: the dual barrel syringe comprises a first diluent chamber holding a first diluent, a second diluent chamber holding a second diluent, a first port fluidically coupled to the first diluent chamber, and a second port fluidically coupled to the second diluent chamber; and the dual chamber mixing cartridge comprises a first powder chamber holding a first powder, a second powder chamber holding a second powder, a first cartridge port fluidically coupled to the first powder chamber, and a second cartridge port fluidically coupled to the second powder chamber, and a plurality of mixing masses positioned within the first powder chamber and the second powder chamber; and advancing a plunger within at least one of the first diluent chamber or the second diluent chamber to cause the first diluent or the second diluent to enter the first powder chamber or the second powder chamber, respectively; and agitating the plurality of mixing masses within the dual chamber mixing cartridge to dissolve the first powder or the second powder within the respective first diluent or second diluent.
13. The method of any preceding clause, wherein coupling the dual barrel syringe to the dual chamber mixing cartridge comprising coupling a collar to both the dual barrel syringe and the dual chamber mixing cartridge such that the (1) the first port is fluidically coupled to the first cartridge port and (2) the second port is fluidically coupled to the second cartridge port.
14. The method of any preceding clause, wherein coupling the collar to both the dual barrel syringe and the dual chamber mixing cartridge comprises rotating a portion of the collar relative to the dual barrel syringe.
15. The method of any preceding clause, wherein coupling the collar to both the dual barrel syringe and the dual chamber mixing cartridge comprises rotating a portion of the collar relative to the dual chamber mixing cartridge.
16. The method of any preceding clause, wherein coupling the collar to both the dual barrel syringe and the dual chamber mixing cartridge comprises rotating a portion of the collar relative to both the dual barrel syringe and the dual chamber mixing cartridge.
17. A method of mixing component materials, the method comprising: coupling a dual barrel syringe to a dual chamber mixing cartridge with a collar, wherein: the dual barrel syringe comprises a first diluent chamber holding a first diluent, a second diluent chamber holding a second diluent, a first port fluidically coupled to the first diluent chamber, a second port fluidically coupled to the second diluent chamber; the dual chamber mixing cartridge comprises a first powder chamber holding a first powder, a second powder chamber holding a second powder, a first cartridge port fluidically coupled to the first powder chamber, and a second cartridge port fluidically coupled to the second powder chamber; and the collar fluidically couples (1) the first port and the first cartridge port and (2) the second port and the first cartridge port, wherein the collar is rotatable relative to the first and second ports and first and second cartridge ports to couple the dual chamber mixing cartridge to the dual barrel syringe; advancing a plunger within at least one of the first diluent chamber or the second diluent chamber to cause the first diluent or the second diluent to enter the first powder chamber or the second powder chamber through the collar, respectively; and agitating the dual chamber mixing cartridge to dissolve the first powder or the second powder within the respective first diluent or second diluent.
18. The method of any preceding clause, wherein coupling the collar to both the dual barrel syringe and the dual chamber mixing cartridge comprises rotating a portion of the collar relative to the dual barrel syringe.
19. The method of any preceding clause, wherein coupling the collar to both the dual barrel syringe and the dual chamber mixing cartridge comprises rotating a portion of the collar relative to the dual chamber mixing cartridge.
20. The method of any preceding clause, wherein coupling the collar to both the dual barrel syringe and the dual chamber mixing cartridge comprises rotating a portion of the collar relative to both the dual barrel syringe and the dual chamber mixing cartridge.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components. Moreover, embodiments as provided herein may assist in maintaining sterility and/or constituent material integrity, while improving ease of mixing and delivery.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A syringe system comprising:
a dual barrel syringe comprising a first diluent chamber, a second diluent chamber, a first port fluidically coupled to the first diluent chamber, and a second port fluidically coupled to the second diluent chamber;
a dual chamber mixing cartridge configured to mount to the dual barrel syringe and comprising a first powder chamber, a second powder chamber, a first cartridge port fluidically coupled to the first powder chamber, and a second cartridge port fluidically coupled to the second powder chamber; and
a plurality of mixing masses positioned within the first powder chamber and the second powder chamber.

2. The syringe system of claim 1, wherein the dual barrel syringe and the dual chamber mixing cartridge couple to one another via a quarter turn coupling structure.

3. The syringe system of claim 1, further comprising a collar configured to receive and fluidically couple (1) the first port and the first cartridge port and (2) the second port and the first cartridge port.

4. The syringe system of claim 1, further comprising an applicator configured to mount to the first port and the second port of the dual barrel syringe.

5. A syringe system comprising:
a dual barrel syringe comprising a first diluent chamber, a second diluent chamber, a first port fluidically coupled to the first diluent chamber, and a second port fluidically coupled to the second diluent chamber;
a dual chamber mixing cartridge comprising a first powder chamber, a second powder chamber, a first cartridge port fluidically coupled to the first powder chamber, and a second cartridge port fluidically coupled to the second powder chamber; and
a collar configured to receive and fluidically couple (1) the first port and the first cartridge port and (2) the second port and the first cartridge port, wherein the collar is rotatable relative to the first and second ports and first and second cartridge ports to couple the dual chamber mixing cartridge to the dual barrel syringe.

6. The syringe system of claim 5, wherein:
the dual barrel syringe comprises a first coupling member and a second coupling member; and
the collar comprises a first tab configured to be engaged to the first coupling member and a second tab configured to be engaged to the second coupling member.

7. The syringe system of claim 5, wherein
the dual chamber mixing cartridge comprises a first cartridge coupling member and a second cartridge coupling member; and
the collar comprises a first tab configured to be engaged to the first cartridge coupling member and a second tab configured to be engaged to the second cartridge coupling member.

8. The syringe system of claim 5, wherein the collar comprises an internal body and an external housing, wherein the external housing rotates relative to the internal body.

9. The syringe system of claim 5, wherein the collar comprises a plurality of ramped engagement tabs for engaging with a corresponding coupling members of the dual barrel syringe and/or the dual chamber mixing cartridge.

10. The syringe system of claim 5, further comprising a plurality of mixing masses positioned within the first powder chamber and the second powder chamber.

11. The syringe system of claim 5, further comprising an applicator configured to mount to the first port and the second port of the dual barrel syringe.

12. A method of mixing constituent materials, the method comprising:
coupling a dual barrel syringe to a dual chamber mixing cartridge, wherein:
the dual barrel syringe comprises a first diluent chamber holding a first diluent, a second diluent chamber holding a second diluent, a first port fluidically coupled to the first diluent chamber, and a second port fluidically coupled to the second diluent chamber; and
the dual chamber mixing cartridge comprises a first powder chamber holding a first powder, a second powder chamber holding a second powder, a first cartridge port fluidically coupled to the first powder chamber, and a second cartridge port fluidically coupled to the second powder chamber, and a plurality of mixing masses positioned within the first powder chamber and the second powder chamber; and
advancing a plunger within at least one of the first diluent chamber or the second diluent chamber to cause the first diluent or the second diluent to enter the first powder chamber or the second powder chamber, respectively; and
agitating the plurality of mixing masses within the dual chamber mixing cartridge to dissolve the first powder or the second powder within the respective first diluent or second diluent.

13. The method of claim 12, wherein coupling the dual barrel syringe to the dual chamber mixing cartridge comprising coupling a collar to both the dual barrel syringe and the dual chamber mixing cartridge such that the (1) the first port is fluidically coupled to the first cartridge port and (2) the second port is fluidically coupled to the second cartridge port.

14. The method of claim 13, wherein coupling the collar to both the dual barrel syringe and the dual chamber mixing cartridge comprises rotating a portion of the collar relative to the dual barrel syringe.

15. The method of claim 13, wherein coupling the collar to both the dual barrel syringe and the dual chamber mixing cartridge comprises rotating a portion of the collar relative to the dual chamber mixing cartridge.

16. The method of claim 13, wherein coupling the collar to both the dual barrel syringe and the dual chamber mixing cartridge comprises rotating a portion of the collar relative to both the dual barrel syringe and the dual chamber mixing cartridge.

17.-20. (canceled)
